(19) United States
(12) Patent Application Publication
(10) Pub. No.: US 2013/0296280 A1
(43) Pub. Date: Nov. 7, 2013

(54) EUTECTIC MIXTURE COMPRISING CELECOXIB AND POLOXAMER

(71) Applicants: Eun Seok Park, Suwon-si (KR); Yun Seok Rhee, Jinju-si (KR); Chun Woong Park, Suwon-si (KR); Tack Oon Oh, Daejeon (KR); Ju Young Kim, Seoul (KR); Jung Myung Ha, Ulsan (KR); Ji Ho Shin, Suwon-si (KR)

(72) Inventors: Eun Seok Park, Suwon-si (KR); Yun Seok Rhee, Jinju-si (KR); Chun Woong Park, Suwon-si (KR); Tack Oon Oh, Daejeon (KR); Ju Young Kim, Seoul (KR); Jung Myung Ha, Ulsan (KR); Ji Ho Shin, Suwon-si (KR)

(21) Appl. No.: 13/677,738

(22) Filed: Nov. 15, 2012

(30) Foreign Application Priority Data
May 4, 2012 (KR) 10-2012-0047766

(51) Int. Cl. A61K 47/10

(52) U.S. Cl. CPC A61K 47/10
USPC 514/158

(57) ABSTRACT

The present invention relates to a eutectic mixture wherein poloxamer is added to the poorly soluble drug celecoxib to significantly increase the solubility and bioavailability of celecoxib.

### CROSS-REFERENCE TO RELATED APPLICATIONS

This application claims the benefit under 35 U.S.C. §119(a) of Korean Patent Application No. 10-2010-0047766 filed on Mar. 3, 2012, in the Korean Intellectual Property Office, the entire disclosure of which is incorporated herein by reference for all purposes.

### BACKGROUND

1. Technical Field

The present invention relates to a eutectic mixture comprising celecoxib or a pharmaceutically acceptable salt thereof and poloxamer.

2. Description of Related Art

Celecoxib is currently frequently used as an inhibitor of cyclooxygenase-2 and is being marketed as an anti-inflammatory drug under the trade name Celebrex®. Methods for preparing celecoxib are disclosed in U.S. Pat. No. 5,466,823 and U.S. Pat. No. 5,892,053. PCT Patent Publication No. WO 00/32189 discloses that celecoxib has a crystal morphology that tends to form long cohesive needles.

Celecoxib has very low solubility in aqueous media, and thus is not readily dissolved and dispersed when administered orally, for example in tablet or capsule form. For this reason, the bioavailability of celecoxib is as low as about 22-40% (Drug Metab Dispos. 2000:28:308-314). In addition, celecoxib has a relatively high dose and rapid absorption requirements further increasing difficulties of providing a sufficient therapeutically effective dose.

In order to solve the above-described problems, various techniques for solubilizing celecoxib have been used in the prior art.

U.S. Pat. No. 5,993,858 discloses a formulation for increasing the bioavailability of a water-soluble drug. The formulation includes an oil or other lipid material, a surfactant, and a hydrophilic co-surfactant and was designed so as to form an emulsion, or a microemulsion in any case, generally when exposed to gastrointestinal fluids. However, the self-emulsifying formulation still has a tendency to precipitate and/or crystallize in gastrointestinal fluids, and thus is unsuitable as a formulation of celecoxib.

In addition, inclusion compounds or solid dispersion techniques were used for solubilization of celecoxib, but their effects on solubility improvement were insignificant, and thus the bioavailability of celecoxib was barely improved.

Generally, drugs show faster dissolution and more rapid onset of action in a semi-solid or liquid state than in a powder state. In view of this fact, there have been many efforts to prepare liquid formulations of celecoxib. However, to prepare a liquid formulation of celecoxib, a large amount of a solvent is required (Drug Discoveries & Therapeutics. 2010; 4(6):459-471. Acta Poloniae Pharmaceutica—drug research 61(5):335-341). In addition, the prepared liquid formulation of celecoxib is significantly influenced by changes in surrounding environmental conditions such as temperature and humidity.

Accordingly, the present inventor has made extensive efforts to maximize the bioavailability of celecoxib by improving the solubility, and as a result, has found that poloxamer forms a eutectic mixture with celecoxib to significantly improve the water solubility of celecoxib, and a eutectic mixture of celecoxib and poloxamer shows physical and chemical stability against environmental changes after preparation, thereby completing the present invention.

### SUMMARY

It is an object of the present invention to provide a celecoxib-poloxamer eutectic mixture, which is prepared by adding poloxamer to the poorly soluble drug celecoxib and has significantly increased solubility and bioavailability.

In order to accomplish the above object, the present invention provides a eutectic mixture comprising celecoxib or a pharmaceutically acceptable salt thereof and poloxamer.

As used herein, the term “celecoxib” means a compound having the chemical name 4-[5-(4-methylphenyl)-3-(trifluoromethyl)-1H-pyrazol-1-yl]benzenesulfonamide and having a structure represented by the following formula 1. Celecoxib has selective cyclooxygenase-2 inhibitory activity and is generally used as an anti-inflammatory agent or for the prevention and treatment of cyclooxygenase-2-mediated disorders.

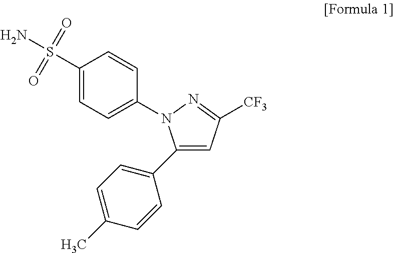 [SMILES: Cc1ccc(-c2cc(C(F)(F)F)nn2-c2ccc(S(N)(=O)=O)cc2)cc1]

As used herein, the term “pharmaceutically acceptable salt” means a salt prepared according to a conventional method known to those skilled in the art. Specifically, the pharmaceutically acceptable salts include, but are not limited to, salts derived from pharmacologically or physiologically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfonic, tartaric, acetic, citric, formic, benzoic, malonic, naphthalene-2-sulfonic and benzenesulfonic acids. Salts derived from suitable bases include alkali metals, for example, sodium or potassium, and alkaline earth metals, for example, magnesium.

Generally, rapid release or dissolution type pharmaceutical formulations can provide effective prevention and treatment within a short time compared to general dosage forms or sustained-release dosage forms. For example, for the alleviation and treatment of acute pain, celecoxib having rapid dissolution properties will be useful for providing rapid pain relief. However, formulating celecoxib encountered several problems as follows.

Celecoxib has very low solubility in aqueous media, and thus is not readily dissolved and dispersed in the gastrointestinal tract when formulated in tablet or capsule form. Also, celecoxib has electrostatic and cohesive properties, low bulk density, low compressibility and poor flowability, which make it difficult to prepare a uniform composition. As described above, celecoxib has low solubility, which makes it difficult to prepare a uniform mixture, and it is not readily released and dissolved. Thus, it has low bioavailability.

In order to solve the above problems, the present invention provides a eutectic mixture having significantly improved solubility by adding poloxamer to celecoxib or a pharmaceutically acceptable salt thereof.

As used herein, the term “poloxamer” means a block copolymer of poly(oxyethylene) and poly(oxypropylene) and has a structure represented by the following formula 2.

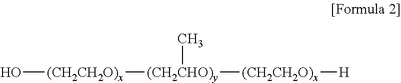

wherein x is 2 to 125, and y is 5 to 235, provided that 2x is 10-80% of 2x+y and further the number-average molecular weight of the poloxamer nonionic surfactant is 1,100-14,600. The poloxamer is commercially easily available from, for example, BASF Corporation, Performance Products, etc. Preferably, the poloxamer may be poloxamer 188 or poloxamer 407.

The poloxamer forms a eutectic mixture with celecoxib at room temperature to significantly increase the water solubility of celecoxib, thus increasing the bioavailability of celecoxib.

Preferably, the weight ratio of poloxamer:celecoxib may be 3:7 to 6:4. More preferably, the eutectic mixture according to the present invention may comprise 1.5 parts by weight of poloxamer per part by weight of celecoxib.

In one example of the present invention, the observation of phase changes as a function of the ratio of celecoxib to poloxamer was carried out (FIG. 1). As a result, as can be seen in FIG. 1, in region I, poloxamer and celecoxib were all present in the solid state, and in region II, poloxamer was in the solid state, and celecoxib was in the liquid state. In region ill, poloxamer was in the liquid state, and celecoxib was in the solid state, and in region IV, poloxamer and celecoxib were all in the liquid state. The region in which celecoxib becomes liquid was observed at a temperature higher than 150° C. if celecoxib was present alone (100%), indicating that celecoxib exists in crystalline form at room temperature. This suggests that, if celecoxib is administered alone at room temperature, the bioavailability thereof is necessarily reduced due to the crystalline form thereof. However, it was found that, when poloxamer was added to celecoxib, poloxamer and celecoxib formed a eutectic mixture, which had significantly increased solubility and thus could be maintained in a liquid or semi-solid state even at room temperature. Particularly, it was found that, when celecoxib and poloxamer were mixed at a weight ratio of 4:6, they formed a eutectic mixture at room temperature.

In another example of the present invention, a PXRD pattern was measured as a function of the ratio of celecoxib to poloxamer. As a result, when the weight ratio of celecoxib was more than 80% or less than 20% (that is, the weight ratio of poloxamer was less than 20% or more than 80%), the crystallinity of the mixture significantly increased (FIG. 2). Thus, it could be seen that a mixture containing poloxamer and celecoxib at a weight ratio ranging from 3:7 to 6:4 has little or no crystallinity.

As used herein, the term “little or no crystallinity” means particles lacking a regular crystalline structure. Celecoxib particles having little or no crystallinity have an advantage in that they are degraded by energy lower than that for celecoxib particles having similar sizes, and thus can show increased solubility and dissolution rate.

The eutectic mixture according to the present invention can be prepared by any suitable method known to those skilled in the art. For example, a method for preparing the eutectic mixture may comprise the steps of:

(a) dissolving celecoxib or a pharmaceutically acceptable salt thereof and poloxamer in a liquid solvent to form a solution; and (b) cooling the solution to form a celecoxib-poloxamer eutectic mixture.

The eutectic mixture may be in a liquid or semi-solid form. In addition, the preparation method may further comprise a step of drying the eutectic mixture.

Examples of a suitable liquid solvent that may be used to the celecoxib-poloxamer eutectic mixture include any pharmaceutically acceptable solvents in which celecoxib can be dissolved. Moreover, heating and stirring may be used to facilitate the dissolution of celecoxib.

Also, the liquid solvent may include a non-solvent portion, for example, a co-solvent selected from among water, alcohol, polyethylene glycol, ethyl caprylate, propylene glycol laurate, diethyl glycol monoethylether, tetraethylene glycol dimethyl ether, triethylene glycol dimethyl ether, and triethylene glycol monoethyl ether. Preferably, polyethylene glycol may be used as the co-solvent. When polyethylene glycol is used, there is an advantage in that it is easier to fill the eutectic mixture of the present invention into a soft capsule.

The eutectic mixture of the present invention, prepared according to the above method or any method known to those skilled in the art, may be administered without formulation or may be administered as a simple suspension in water or other pharmaceutically acceptable liquids.

Alternatively, the eutectic mixture of the present invention may additionally be formulated with one or more pharmaceutically acceptable excipients.

As used herein, “excipient” means any material, which is used as a carrier or medium for delivery of celecoxib or is added to a pharmaceutical composition to improve the handling or storage properties of the composition or makes it easy to prepare a unit dosage composition into dosage forms such as capsules or tablets, which are suitable for oral administration. The excipients that are used in the present invention may be diluents, disintegrants, binders, adhesives, wetting agents, lubricants, aromatics, surfactants, and inclusion compounds. Preferably, surfactants or inclusion compounds may be used.

As used herein, the term “surfactant” means a material that absorbs to a solution to reduce the surface tension. The surfactant that may be used in the present invention may be any surfactant which is generally used in the art. For example, the surfactant may be any one or more of tween 80, span 80, cremophore RH 40, and cremophore EL.

As used herein, the term “inclusion compound” means a complex compound formed by enclosure of a certain chemical species (guest) by another compound (host) having a molecular space adapted for the guest with respect to the size and shape. The inclusion compound that may be used in the present invention may be any inclusion compound which is generally used in the art. For example, it may be cyclodextrin.

The eutectic mixture of the present invention may be administered by any general route, as long as it can reach a target tissue. Specifically, the composition of the present invention may be administered intraperitoneally, intravenously, intramuscularly, subcutaneously, intradermally, orally, intranasally, intrapulmonarily or intrarectally, but is not limited thereto. In addition, the pharmaceutical composition of the present invention may be administered using any system capable of delivering the active ingredient to target cells.

According to the present invention, poloxamer is added to the poorly soluble drug celecoxib to form a eutectic mixture, thereby significantly increasing the solubility and bioavailability of celecoxib.

### BRIEF DESCRIPTION OF THE DRAWINGS

FIG. 1 is a graphic diagram showing the phase change of celecoxib, measured in one example of the present invention.

FIG. 2 is a graphic diagram showing the PXRD pattern of celecoxib, measured in one example of the present invention.

FIG. 3 is a graphic diagram showing a dissolution pattern as a function of the kind of additive, measured in one example of the present invention.

FIG. 4 is a graphic diagram showing the change in viscosity as a function of temperature, measured in one example of the present invention.

### DETAILED DESCRIPTION

Hereinafter, the present invention will be described in further detail with reference to examples. It is to be understood, however, that these examples are for illustrative purposes and are not intended to limit the scope of the present invention.

### Examples 1 to 7

According to the compositions shown in Table 1 below, celecoxib, poloxamer and the like were mixed with each other to prepare eutectic mixtures which were then heated in a water bath at 80° C. or heated directly. Then, the mixtures were homogenized by stirring and cooled at room temperature or lower, thereby preparing celecoxib-poloxamer eutectic mixtures of Examples 1 to 6. In Example 7, celecoxib, poloxamer and tween 80 were mixed with each other, and the mixture was heated in a water bath at 80° C. or heated directly, homogenized by stirring and cooled at room temperature or lower, after which polyethylene glycol was added thereto, thereby preparing a celecoxib-poloxamer eutectic mixture.

TABLE 1

| | Example | Example | Example | Example | Example | Example | Example |
|---|---|---|---|---|---|---|---|
| | 1 | 2 | 3 | 4 | 5 | 6 | 7 |
| Celecoxib | 100 | 100 | 100 | 100 | 100 | 100 | 100 |
| Poloxamer 407 | 150 | 150 | 150 | 150 | 150 | 150 | 150 |
| Polyethylene glycol 400 | 100 | | | | | | 100 |
| Tween 80 | | 100 | | | | | 100 |
| Span 80 | | | 100 | | | | |
| Cremophore EL | | | | 100 | | | |
| Cremophore RH40 | | | | | 100 | | |
| HP-β-CD | | | | | | 100 | |
| Total weight (mg) | 350 | 350 | 350 | 350 | 350 | 350 | 450 |

### Comparative Examples 1 and 2

In order to examine the dissolution pattern of a mixture comprising celecoxib alone without poloxamer and a dissolution pattern as a function of the content of poloxamer, mixtures having the compositions shown in Table 2 below were prepared in the same manner as Examples 1 to 6.

TABLE 2

| | Comparative | Comparative |
|---|---|---|
| | Example 1 | Example 2 |
| Celecoxib | 100 | 100 |
| Poloxamer 407 | | 100 |
| Polyethylene glycol 400 | | 100 |
| Tween 80 | 200 | 100 |
| Span 80 | | |
| Cremophore EL | | |
| Cremophore RH40 | | |
| HP-β-CD | | |
| Total weight (mg) | 300 | 400 |

### Test Example 1

### Observation of Phase Change as a Function of the Ratio of Celecoxib

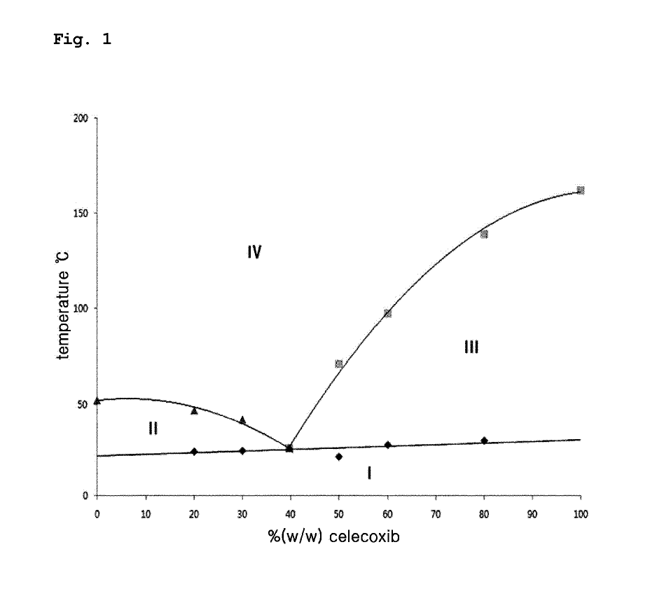

The analysis of phase changes was carried out by DSC while changing the weight ratio of celecoxib:poloxamer from 0% to 100%. The DSC analysis was carried out using Exstar 600 (Seiko) at a temperature ranging from 0 to 200° C. at a heating rate of 10° C./min. The results of the analysis are shown in FIG. 1.

As can be seen in FIG. 1, the mixtures of celecoxib and poloxamer showed four phases. In region I, poloxamer and celecoxib were all present in the solid state, and in region II, poloxamer was in the solid state, and celecoxib was in the liquid state. In region ill, poloxamer was in the liquid state, and celecoxib was in the solid state, and in region IV, poloxamer and celecoxib were all in the liquid state. Particularly, when the mixture had a celecoxib content of about 40 wt %, eutectic temperature thereof was observed at the range of room temperature. Thus, it was found that, when poloxamer is added to celecoxib, they form a eutectic mixture, which has significantly increased solubility and can be maintained in a liquid or semi-solid state even at room temperature.

### Test Example 2

### PXRD Pattern as a Function of the Ratio of Celecoxib

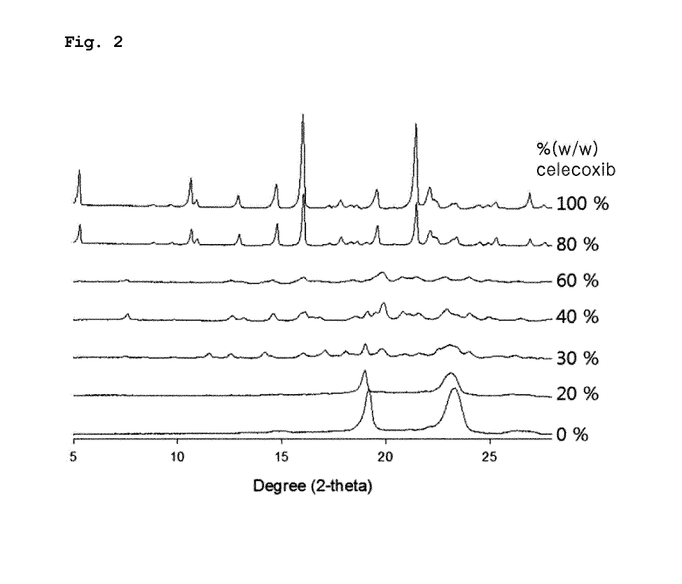

In order to observe the change in the crystalline form of celecoxib as a function of the weight ratio of celecoxib to poloxamer, the PXRD patterns of the mixtures were measured using D8 focus (Bruker AXS). The measurement was carried out at 2-theta degree of 3-40° at a rate of 1°/min. The results of the measurement are shown in FIG. 2.

As can be seen in FIG. 2, the weight ratio of celecoxib was more than 80% or less than 20% (that is, the weight ratio of poloxamer was less than 20% or more than 80%), the crystallinity of the mixture significantly increased. In addition, it could be seen that, when the weight ratio of poloxamer:celecoxib was in the range of 3:7 to 6:4, the mixture had little or no crystallinity. In other words, it could be seen that, when celecoxib and poloxamer are used in the above weight ratio, celecoxib has little or no crystallinity, and thus the solubility and bioavailability thereof could be improved.

### Test Example 3

### Dissolution Pattern as a Function of the Kind of Additive

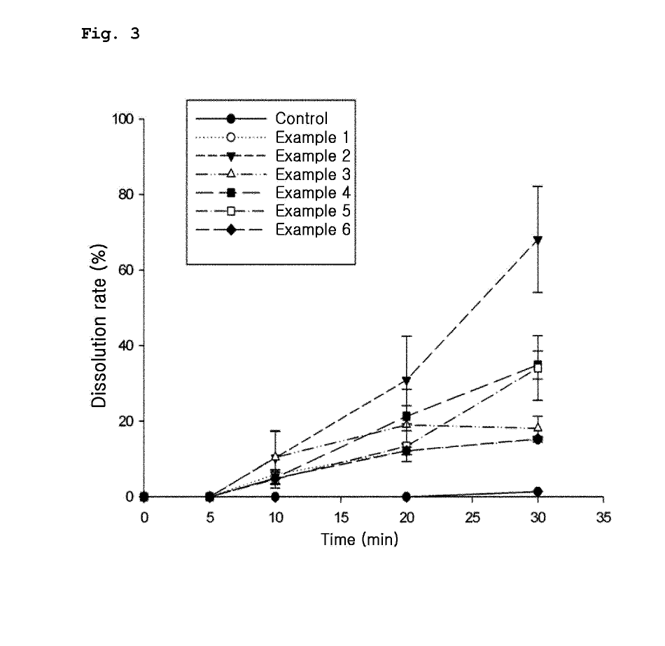

In order to examine the dissolution patterns of the mixtures of Examples 1 to 6, a dissolution test were carried out in accordance with the paddle method (dissolution test method 2) described in the Korean Pharmacopoeia. As a control, commercially available Celebrex (Pfizer Korea) was used. Specifically, the dissolution test was carried out in 900 mL of a solution (pH 1.2) at 100 rpm. The dissolution patterns were analyzed using HPLC-UV, and the results of the analysis are shown in FIG. 3.

As can be seen in FIG. 3, Celebrex used as the control showed little or no dissolution even after 30 minutes. In contrast, the mixtures of Examples 1 to 6 according to the present invention showed a dissolution rate of 20-70% at 30 minutes after the start of the test. Thus, it was found that the addition of poloxamer to celecoxib significantly improves the dissolution rate of celecoxib.

### Test Example 4

### Change in Viscosity as a Function of Temperature

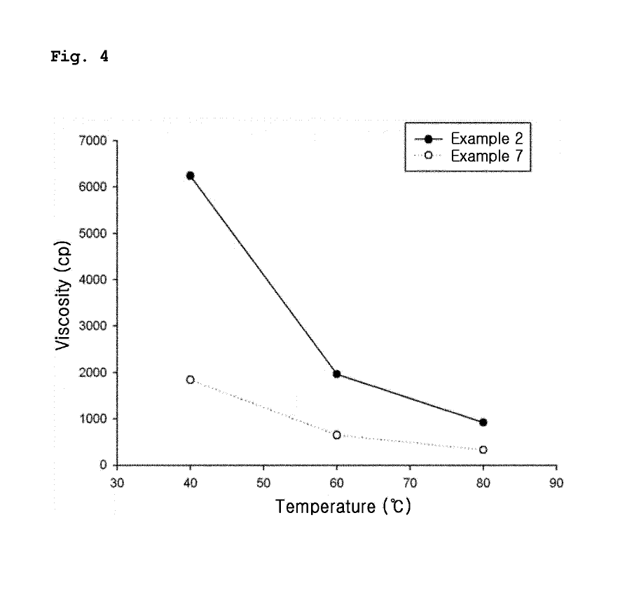

In order to examine the change in viscosity as a function of temperature, the viscosities of the mixtures of Examples 2 and 7 were measured. Specifically, the viscosities were measured using a DVII+viscometer (Brookfield) equipped with a #63 spindle at a speed of 12 rpm. The results of the measurement are shown in FIG. 4.

As can be seen in FIG. 4, the eutectic mixtures of Examples 2 and 7 all showed a significant decrease in viscosity with increasing temperature.

1. A eutectic mixture comprising celecoxib or a pharmaceutically acceptable salt thereof and poloxamer.

2. The eutectic mixture of claim 1, wherein the poloxamer is poloxamer 188 or poloxamer 407.

3. The eutectic mixture of claim 1, wherein the weight ratio of the poloxamer to the celecoxib is 3:7 to 6:4.

4. The eutectic mixture of claim 1, wherein the eutectic mixture comprises 1.5 parts by weight of the poloxamer per part by weight of the celecoxib.

5. The eutectic mixture of claim 1, wherein the eutectic mixture comprises a surfactant or an inclusion compound.

6. The eutectic mixture of claim 5, wherein the inclusion compound is beta-cyclodextrin.

7. The eutectic mixture of claim 4, wherein the eutectic mixture comprises a co-solvent.

8. The eutectic mixture of claim 7, wherein the co-solvent is polyethylene glycol.

9. The eutectic mixture of claim 1, wherein the poloxamer is poloxamer 407; and the eutectic mixture comprises 1.5 parts by weight of the poloxamer per part by weight of the celecoxib.

10. The eutectic mixture of claim 9, wherein the eutectic mixture is obtained by forming a homogenous mixture of a co-solvent, the poloxamer and the celecoxib, and cooling the homogenous mixture.
